(19) United States
(12) Patent Application Publication
(10) Pub. No.: US 2026/0350804 A1
(43) Pub. Date: Nov. 5, 2026

(54) System for Cell Interaction Monitoring

(71) Applicant: IMEC VZW, Leuven (BE)

(72) Inventors: Chengxun LIU, Heverlee (BE); Ziduo LIN, Heverlee (BE); Yuqian LI, Heverlee (BE); Murali JAYAPALA, Kumtich (BE); Zhenxiang LUO, Leuven (BE)

(21) Appl. No.: 19/673,752

(22) Filed: May 11, 2026

Related U.S. Application Data
(63) Continuation of application No. PCT/EP2025/064520, filed on May 26, 2025.

(30) Foreign Application Priority Data
May 27, 2024 (EP) 24178235.8

(51) Int. Cl. G06T 7/00; G01N 33/483

(52) U.S. Cl. CPC G06T 7/0016; G01N 33/4833; G06T 2207/10028; G06T 2207/30024

(57) ABSTRACT

A system for biological cell monitoring includes a containment unit with multiple discrete confinement locations, each capable of holding at least one biological cell. The system also features a lens-free imaging module for capturing images that simultaneously depict all biological cells within the containment unit. Additionally, a control unit is configured to operate the imaging module to capture a sequence of images over time and to extract features from these images related to the biological cells. This system facilitates detailed analysis of cell features and behaviors over time, providing valuable insights for biological research and applications.

### CROSS-REFERENCE TO RELATED APPLICATION

The present application is a non-provisional patent application which is a national phase entry into the United States of International Application No. PCT/EP2025/064520, filed on May 26, 2025, and which claims priority to European Patent Application No. EP24178235.8, filed May 27, 2024, the contents of both of which are hereby incorporated by reference.

### FIELD OF THE DISCLOSURE

The present disclosure relates to the field of biological cell analysis, specifically to systems and methods for monitoring cells over time.

### BACKGROUND

In the realm of biological research and medical diagnostics, the ability to monitor and analyze cell-stimulus interactions is crucial. These interactions are fundamental to understanding cellular behaviors in response to various stimuli, which can be advantageous for advancements in drug development, disease diagnosis, and therapeutic interventions. Traditionally, cell analysis has been heavily reliant on static imaging techniques. These methods capture single moments in the life cycle of a cell, providing a snapshot that might not accurately reflect the dynamic nature of cellular processes.

One of the primary challenges in this field is the limitation imposed by snapshot imaging. This technique, while useful in certain contexts, often fails to capture the full spectrum of cellular dynamics over time. Cells respond to stimuli in complex ways that can evolve minute by minute, and static images taken at single time points can miss particularly advantageous interactions or changes. This can lead to incomplete or inaccurate data, which might affect the subsequent biological or medical conclusions drawn from such analyses.

Moreover, the reliance on static imaging can be particularly problematic when dealing with processes that unfold over extended periods. For instance, some cellular responses to pharmacological treatments or genetic modifications might not become apparent until several hours after the initial stimulus. If the imaging is performed too early or too late, the opportunity to observe these responses is missed, potentially leading to misinterpretation of a cell's health or behavior.

### SUMMARY

It is an object of embodiments of the present disclosure to enable continuous monitoring and characterization of cells over time to enhance accuracy in cell activity assessment. This objective can be accomplished by a system for biological cell monitoring according to the present disclosure.

In the first aspect, the present disclosure relates to a system for biological cell monitoring, comprising:
a containment unit comprising a plurality of discrete confinement locations, the containment unit being adapted to hold at least one biological cell per confinement location;
a lens-free imaging module for capturing images, each image simultaneously depicting all biological cells within the containment unit; and
a control unit configured to operate the lens-free imaging module so that it captures a sequence of the images over a period of time, and it extracts biological cell features from the images.

In embodiments, the system may be for analyzing group behavior of cells with respect to a stimulus. This allows studying cell-cell interactions and responses.

In embodiments, the confinement in the confinement locations may be achieved by electrical, optical, physical, or acoustic means. These provide flexibility in isolating cells.

In embodiments, the biological cell features may be selected from spatial features and temporal features. This enables capturing both morphological and dynamic cell characteristics.

In embodiments, the biological cell features may be selected from morphology, behaviors, and intra-cell activity. These provide a comprehensive view of the cell state and activity.

In embodiments, the biological cell feature may be a biological cell morphology feature selected from static cell morphology features and dynamic cell morphology features. Morphology is a key indicator of cell type and health.

In embodiments, the static cell morphology features may be selected from cell size, circularity, nucleus/cell size ratio, and optical intensity contrast between different parts of cells. These are relevant markers to characterize cells.

In embodiments, the dynamic cell morphology feature may be selected from cell motility and changes in static cell morphology feature. Temporal changes reveal important cell processes.

In embodiments, the control unit may be further configured to create time-lapse imaging data from the sequence. Time-lapse data captures the progression of cellular events.

In embodiments, the control unit may be further configured to detect an individual biological cell from the biological cell features. Identifying single cells enables tracking them over time.

In embodiments, the control unit may be further configured to identify the detected individual biological cell. Assigning identities allows following specific cells.

In embodiments, the control unit may be further configured to classify the individual biological cell from the biological cell features. Classification enables categorizing cells into functional types.

In embodiments, the classification may be performed by machine learning. Machine learning can handle the complexity of cellular data.

In embodiments, the classification may comprise one or more decision-making method selected from statistical classification techniques, decision trees, rule-based systems, Bayesian methods, ensemble methods, support vector machines, nearest neighbor, clustering techniques, dimensionality reduction techniques, amongst others. These are powerful techniques for cellular classification.

In embodiments, the control unit may be further configured to detect the interaction of the biological cell with a stimulus from an evolution of the biological cell features in the sequence. Analyzing feature changes over time reveals stimulus responses.

In embodiments, the stimulus may be selected from another biological cell, a microstructure, a particle such as a bead, and a chemical such as a drug, a toxin, a protein, or a polynucleotide. These cover a wide range of biologically relevant stimuli.

In embodiments, the stimulus may be another biological cell. Cell-cell interactions mediate many important biological processes.

In embodiments, the stimulus may be a microstructure coated with biological molecules. The cell-microstructure interaction is mediated by the affinity between the biological molecules and cell markers. Therefore, the cell-microstructure interaction patterns can be used to derive the cell marker expressions.

In embodiments, the stimulus may be a bead coated with biological molecules. The cell-bead interaction is mediated by the affinity between the biological molecule and cell markers. Therefore, the cell-bead interaction patterns can be used to derive the cell marker expressions.

In embodiments, the control unit may be further configured to extract interaction patterns from the interactions. Recurring interaction motifs can characterize specific cellular behaviors.

In embodiments, the control unit may be further configured to classify the interaction as following a particular interaction pattern. This enables identifying and quantifying distinct interaction types.

In embodiments, the classification may be performed by machine learning. Machine learning excels at finding patterns in complex interaction data.

In embodiments, the classification may comprise one or more decision-making method selected from statistical classification techniques, decision trees, rule-based systems, Bayesian methods, ensemble methods, support vector machines, nearest neighbor, clustering techniques, dimensionality reduction techniques, amongst others. These techniques can classify interactions based on extracted patterns.

In embodiments, the control unit may be further configured to detect changes in the biological cell features. Detecting feature changes is key to assessing cell responses.

In embodiments, the control unit may be further configured to quantify the changes in the biological cell features. Quantifying the degree of change allows precise comparisons between conditions. In embodiments, the control unit may be configured to quantify the change in the biological cell features resulting from the stimulus. This quantification can be achieved by comparing the sample of interest with appropriate positive and negative control samples. For instance, when evaluating the effect of a stimulus on a particular cell type, the sample to be assessed would include the cells exposed to the stimulus. The positive control sample would include cells known to exhibit a strong response to the stimulus, while the negative control would involve cells that are not expected to respond to the stimulus. The anticipated outcome would typically show the highest level of biological change in the positive control, followed by the sample being assessed, and the lowest change in the negative control. The control unit can then derive a relative value representing the magnitude of the biological change based on the comparison of these three values obtained from the sample of interest and the control samples.

In embodiments, at least one image every 30 seconds, for example at least one image every 20 seconds may be captured during the sequence. High temporal resolution enables detecting rapid cellular events. In some embodiments, at least one image every 10 seconds, every second or even every millisecond may be captured during the sequence (see example 7).

In embodiments, the plurality of discrete confinement locations may be an array of microcavities or wherein the containment unit comprises a microfluidic unit configured for forming an array of droplets, the microcavities or droplets being for biological cell holding. Microfluidics enables high-throughput single-cell analysis.

In embodiments, the droplets may be aqueous droplets either in air or in oil. This allows isolating cells in controlled microenvironments.

In embodiments, the containment unit (e.g., the microfluidic unit) may be adapted for enabling the establishment of different cell-stimuli conditions in different confinement locations amongst the plurality of the containment unit. This enables testing cells under various stimuli in parallel.

In embodiments, the different cell-stimuli conditions may be different biological cell environments, e.g., different stimuli or different stimuli concentration, in different confinement locations amongst the plurality of the containment unit. This allows dose-response analyses.

In embodiments, the different cell-stimuli conditions may be different types and/or different ratio of biological cells in different confinement locations amongst the plurality of the containment unit. This allows analyzing interactions between cell types.

In embodiments, the system may further comprise a fluidic control system for introducing or removing fluids from the containment unit. Fluidic control enables dynamic stimulation of the cells.

In embodiments, the plurality of discrete confinement locations may be at least 20, for example at least 200, for example at least 2000, for example at least 20000, for example at least 200000 locations, and in some cases at least 500000 locations. Higher parallelization increases the throughput and statistical power.

In embodiments, the lens-free imaging module may be configured to perform holographic imaging. Holographic imaging can provide 3D information about the cells. Holographic imaging enables both 2D and 3D cell analysis. In embodiments, the lens-free imaging module may be configured to capture 2D or 3D images depicting all biological cells within the containment unit. For example, it can be configured to capture 3D images depicting all biological cells within the containment unit.

In embodiments of the first aspect, the lens-free imaging system may comprise one or more imagers, i.e., one or more image sensors. Using multiple imagers permits to capture different fields of view or different wavelengths of light simultaneously. This can enhance the system's ability to capture comprehensive data about the sample in a shorter amount of time.

In embodiments, the system may further comprise at least one biological cell in one of the confinement locations. The presence of a cell enables the analysis. In embodiments, a single target biological cell per containment unit is present, in addition to the stimulus which can for instance be one or more other cells. This is often advantageous. In other embodiments, more than one target cell is present per containment unit, in addition to the stimulus. This is advantageous for instance in serial events assays.

Any feature of the first aspect may be as correspondingly described in any other aspects of the present disclosure.

In the second aspect, the present disclosure relates to a method for monitoring biological cells, the method comprising:
a. Arranging biological cells within a containment unit, the containment unit being structured to maintain the biological cells in a plurality of discrete confinement locations;
b. Employing a lens-free imaging module to capture a plurality of images, wherein each image simultaneously depicts all biological cells positioned within the containment unit;
c. Operating the lens-free imaging module to capture a sequence of the images over a period of time; and
d. Extract from the images biological cell features.

In embodiments, step a may include arranging biological cells within the containment unit so that at least two biological cells are in two different discrete confinement locations differing in their cell-stimuli conditions. This enables comparing cell responses to different stimuli.

In embodiments, the different cell-stimuli conditions may be different biological cell environments, e.g., different stimuli or different stimuli concentration. This allows testing the effect of the microenvironment on the cells.

In embodiments, the different cell-stimuli conditions may be different types and/or different ratio of biological cells. This allows studying interactions between cell populations.

Any feature of the first aspect may be as correspondingly described in any other aspects of the present disclosure.

In the third aspect, the present disclosure relates to a computer program comprising instructions to cause the system of any embodiment of the first aspect to execute the steps of the method of any embodiment of the second aspect.

Any feature of the first aspect may be as correspondingly described in any other aspects of the present disclosure.

In the fourth aspect, the present disclosure relates to a computer-readable medium having stored thereon the computer program of the third aspect.

Any feature of the first aspect may be as correspondingly described in any other aspects of the present disclosure.

It is an benefit of embodiments of the present disclosure that comprehensive monitoring of cell-cell interactions over time can be achieved, allowing for a detailed characterization of cellular responses and behaviors throughout the entire duration of a stimulus response. Another benefit of embodiments of the present disclosure is that the integration of lens-free imaging technology permits the observation of a large number of cells simultaneously, which is beneficial for high-throughput cell assays and significantly reduces the complexity and cost associated with traditional imaging systems. Moreover, the use of lens-free imaging allows for monitoring a large number of events with high temporal resolution (e.g., every 10 seconds) due to its large field of view, which is not possible with classical lens-based microscopy. This high temporal resolution is particularly meaningful for capturing fast-paced cell-cell interactions, where features can change within a matter of seconds (e.g., every 30 seconds). Additionally, it is a benefit of embodiments of the present disclosure that cells can be individually encapsulated in microfluidic compartments, which supports the examination of heterogeneous cell populations and precise control over the cellular microenvironment. This encapsulation also prevents the formation of cell aggregates, thereby maintaining the integrity of individual cell assessments. Moreover, it is a benefit of embodiments of the present disclosure that dynamic cellular features, such as motility and morphological changes, can be continuously tracked and quantified, providing insights into cellular processes that are not discernible through static snapshot imaging. Furthermore, it is a benefit of embodiments of the present disclosure that the integration of time-lapse data with image analysis algorithms allows for the real-time detection and classification of cellular events, enhancing the accuracy of cell characterization and the assessment of cellular health and functionality. This capability is particularly beneficial in applications such as drug development and cell therapy, where rapid and accurate cell assessments are beneficial. Additionally, it is a benefit of embodiments of the present disclosure that the system is designed to operate without the need for cell labeling, which simplifies the preparation process and reduces the risk of influencing cell behavior through the introduction of foreign substances. This label-free approach not only streamlines the experimental workflow but also preserves the natural state of the cells, leading to more reliable and reproducible data.

Particular example aspects of the disclosure are set out in the accompanying independent and dependent claims. Features from the dependent claims may be combined with features of the independent claims and with features of other dependent claims as appropriate and not merely as explicitly set out in the claims.

The above and other characteristics, features and advantages of the present disclosure will become apparent from the following detailed description, taken in conjunction with the accompanying drawings, which illustrate, by way of example, the principles of the present disclosure. This description is given for the sake of example only, without limiting the scope of the present disclosure. The reference figures quoted below refer to the attached drawings.

### BRIEF DESCRIPTION OF THE DRAWINGS

FIG. 1 is a schematic view of the system for biological cell monitoring according to an example embodiment.

FIG. 2 is a flowchart illustrating the method for monitoring biological cells using the system according to an example embodiment.

FIG. 3 is a diagram of an example algorithm pipeline suitable for use in the system for biological cell monitoring according to an example embodiment.

In the different figures, the same reference signs refer to the same or analogous elements.

### DETAILED DESCRIPTION

Embodiments of the present disclosure will be described with respect to particular example embodiments and with reference to certain drawings but the scope of the claimed invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the subject matter of the present disclosure.

The terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the disclosure described herein are capable of operation in other sequences than described or illustrated herein.

Moreover, the terms top and over and the like in the description and the claims are used for descriptive purposes and not necessarily for describing relative positions. It is to be understood that the terms so used are interchangeable under appropriate circumstances and that the embodiments of the disclosure described herein are capable of operation in other orientations than described or illustrated herein.

It is to be noticed that the term “comprising”, also used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be interpreted as being limited to devices consisting only of components A and B. It means that with respect to the present disclosure, the only relevant components of the device are A and B. The term “comprising” therefore covers the situation where only the stated features are present and the situation where these features and one or more other features are present. The word “comprising” according to the present disclosure therefore also includes as one embodiment that no further components are present. When the word “comprising” is used to describe an embodiment in this application, it is to be understood that an alternative version of the same embodiment, wherein the term “comprising” is replaced by “consisting of”, is also encompassed within the scope of the present disclosure.

Similarly, it is to be noticed that the term “coupled” should not be interpreted as being restricted to direct connections only. The terms “coupled” and “connected”, along with their derivatives, may be used. It should be understood that these terms are not intended as synonyms for each other. Thus, the scope of the expression “a device A coupled to a device B” should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the present disclosure, various features of the disclosure are sometimes grouped together in a single embodiment, figure, or description thereof for the purpose of streamlining the disclosure and aiding in the understanding of one or more of the various aspects thereof. This method of disclosure, however, is not to be interpreted as reflecting an intention that the claimed invention requires more features than are expressly recited in each claim. Rather, inventive aspects may lie in less than all features of a single foregoing disclosed embodiment. Thus, the claims following the detailed description are hereby expressly incorporated into this detailed description, with each claim standing on its own as a separate embodiment of the present disclosure.

Furthermore, while some embodiments described herein include some but not other features included in other embodiments, combinations of features of different embodiments are meant to be within the scope of the present disclosure, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the instructions for carrying out such a method or element of a method forms a means for carrying out the method or element of a method. Furthermore, an element described herein of an apparatus embodiment is an example of a means for carrying out the function performed by the element for the purpose of carrying out subject matter of the present disclosure.

In the description provided herein, numerous specific details are set forth. However, it is understood that embodiments of the present disclosure may be practiced without these specific details. In other instances, well-known methods, structures and techniques have not been shown in detail in order not to obscure an understanding of this description.

The following terms are provided solely to aid in the understanding of the present disclosure.

As used herein, and unless otherwise specified, the term “biological cell” refers to any living cell, including but not limited to mammalian cells, bacterial cells, yeast cells, plant cells, and insect cells. Examples of biological cells include fibroblasts, epithelial cells, endothelial cells, neurons, stem cells, immune cells, cancer cells, and genetically engineered cells.

As used herein, and unless otherwise specified, the term “containment unit” refers to a device or apparatus capable of holding and confining biological cells in discrete locations. The containment unit can be a microfluidic device, a multi-well plate, a microarray, or any other suitable container that allows for the separation and confinement of individual cells or small groups of cells. Examples of containment units include microfluidic chips with microcavities or droplet-generating capabilities, 96-well plates, and microarrays with microwells. In embodiments, the confinement in the confinement locations may be achieved by electrical, optical, physical, or acoustic means, providing flexibility in isolating cells.

As used herein, and unless otherwise specified, the term “confinement location” refers to a specific area within the containment unit where one or more biological cells can be held and confined. Confinement locations can be microcavities, droplets, microwells, or any other suitable structure that allows for the isolation and confinement of individual cells or small groups of cells. In embodiments, the microwell or microcavity may have a width of from 5 to 500 μm and an height to width ratio of from 1 to 2. Examples of confinement locations include microcavities in a microfluidic chip, aqueous droplets in air or oil, and microwells in a microarray.

As used herein, and unless otherwise specified, the term “lens-free imaging module” refers to an imaging device capable of capturing images of biological cells without the use of a conventional lens. The lens-free imaging module can utilize various techniques such as holography, shadow imaging, or diffraction-based imaging to capture images of cells. Examples of lens-free imaging modules include digital inline holographic microscopes, shadow imaging devices, and diffraction-based imaging systems. Holographic microscopes can be used.

As used herein, and unless otherwise specified, the term “control unit” refers to a device or system capable of controlling the operation of the lens-free imaging module and processing the captured images to extract biological cell features. The control unit can be a computer, a microcontroller, or any other suitable processing device. Examples of control units include personal computers, embedded systems, and dedicated image processing units. The control unit must be configured to operate the lens-free imaging module so that it captures a sequence of the images over a period of time, and it extracts biological cell features from the images.

As used herein, and unless otherwise specified, the term “biological cell features” refers to any observable or measurable characteristics of biological cells that can be extracted from the captured images. Biological cell features can include spatial features, such as morphology or size, and temporal features, such as behavior or intra-cell activity. Examples of biological cell features include cell size, circularity, nucleus/cell size ratio, optical intensity contrast between different parts of cells, cell motility, and changes in static cell morphology features over time.

As used herein, and unless otherwise specified, the term “stimulus” refers to any external factor or substance that can influence the behavior, morphology, or activity of biological cells. Stimuli can be physical, chemical, or biological in nature. Examples of stimuli include other biological cells, particles such as beads, and chemicals such as drugs, toxins, proteins, or polynucleotides.

As used herein, and unless otherwise specified, the term “interaction pattern” refers to a specific way in which biological cells interact with each other or with a stimulus. Interaction patterns can be characterized by changes in biological cell features over time or by the spatial arrangement of cells relative to each other or the stimulus. Examples of interaction patterns include attraction, repulsion, aggregation, dispersion, and changes in morphology or activity in response to a stimulus.

The subject matter of the present disclosure will now be described by a detailed description of several example embodiments. It is clear that other embodiments of the present disclosure can be configured according to the knowledge of persons skilled in the art without departing from the technical teaching of the present disclosure, the scope of the invention claimed being limited only by the terms of the appended claims. \

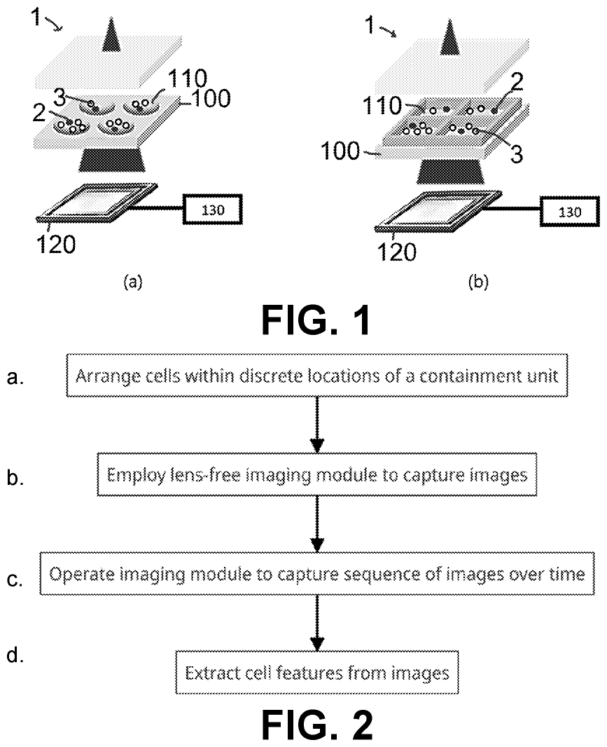

In the first aspect, and as depicted in FIG. 1, the present disclosure relates to a system (1) for biological cell (2) monitoring, comprising:
a containment unit (100) comprising a plurality of discrete confinement locations (110), the containment unit (100) being adapted to hold at least one biological cell (2) per confinement location (110);
a lens-free imaging module (120) for capturing images, each image simultaneously depicting all biological cells (2) within the containment unit (100); and
a control unit (130) configured to operate the lens-free imaging module (120) so that it captures a sequence of the images over a period of time, and it extracts biological cell (2) features from the images.

FIG. 1 illustrates two examples of systems according to the first aspect. the hardware architecture of the imaging approach for monitoring biological cell (2) interactions. This figure is divided into two parts, labeled (a) and (b), each depicting a different configuration of a lens-free imaging (LFI) system (1) used in embodiments of the present disclosure.

In part (a) of FIG. 1, the LFI system (1) is shown with a light source providing LFI illumination directed towards a substrate containing cells (2) encapsulated within microdroplets (220). These microdroplets (220) are depicted as small, rounded compartments (110) on the substrate, each holding a group of cells (2), which are represented in black or white to indicate different cell types. Below the substrate, an imager of the lens-free imaging module (120) is positioned to capture the images transmitted through the substrate. The imager is connected to a control unit (130), which processes the images to extract data on cell features and interactions as described in the present disclosure.

Part (b) of FIG. 1 displays a similar setup with the primary difference being the type of cell containment (110). Here, cells (2) are contained within micro wells (210) instead of microdroplets (220). These micro wells (210) are integrated into the substrate in a grid-like pattern, each well holding a group of cells (2). The same LFI illumination and imaging setup is used here, with the imager positioned below the substrate to capture the images for analysis.

Both configurations utilize the lens-free imaging technique to allow for a broad field of view and high-resolution imaging of multiple cell-cell interaction events simultaneously, which is beneficial for the effective monitoring and analysis of cell behaviors and responses in real-time. The control unit (130) depicted in both parts of the figure is beneficial for processing the captured images to extract and analyze cell features over time, enabling the comprehensive characterization of cell interactions and dynamics.

In embodiments, the system (1) may be for analyzing group behavior of cells (2) with respect to a stimulus, allowing the study of cell-cell interactions and responses.

In embodiments, the confinement in the confinement locations (110) may be achieved by electrical, optical, physical, or acoustic means, providing flexibility in isolating cells (2).

In embodiments, the biological cell (2) features may be selected from spatial features and temporal features, enabling the capture of both morphological and dynamic cell characteristics. The biological cell (2) features may be selected from morphology, behaviors, and intra-cell activity, providing a comprehensive view of the cell state and activity.

In embodiments, the biological cell (2) feature may be a biological cell (2) morphology feature selected from static cell morphology features and dynamic cell morphology features, as morphology is a key indicator of cell type and health. The static cell morphology features may be selected from cell size, circularity, nucleus/cell size ratio, and optical intensity contrast between different parts of cells. The dynamic cell morphology feature may be selected from cell motility and changes in static cell morphology feature.

In embodiments, the control unit (130) may be further configured to create time-lapse imaging data from the image sequence, as time-lapse data captures the progression of cellular events. The control unit (130) may be further configured to detect an individual biological cell (2) from the biological cell (2) features, enabling tracking of single cells over time. The control unit (130) may be further configured to identify the detected individual biological cell (2), allowing specific cells to be followed. The control unit (130) may be further configured to classify the individual biological cell (2) from the biological cell (2) features, enabling categorization of cells into functional types. The classification may be performed by machine learning to handle the complexity of cellular data. The classification may comprise one or more decision-making method selected from statistical classification techniques, decision trees, rule-based systems, Bayesian methods, ensemble methods, support vector machines, nearest neighbor, clustering techniques, dimensionality reduction techniques, amongst others.

In embodiments, the control unit (130) may be further configured to detect the interaction of the biological cell (2) with a stimulus from an evolution of the biological cell (2) features in the image sequence, as analyzing feature changes over time reveals stimulus responses. The stimulus may be selected from another biological cell (2), a microstructure, a particle such as a bead, and a chemical such as a drug, a toxin, a protein, or a polynucleotide. The stimulus may be another biological cell (2).

In embodiments, the stimulus may be a microstructure coated with biological molecules. The cell-microstructure interaction is mediated by the affinity between the biological molecules and cell markers. Therefore, the cell-microstructure interaction patterns can be used to derive the cell marker expressions.

In embodiments, the stimulus may be a bead coated with biological molecules. The cell-bead interaction is mediated by the affinity between the biological molecule and cell markers. Therefore, the cell-bead interaction patterns can be used to derive the cell marker expressions.

In embodiments, the control unit (130) may be further configured to extract interaction patterns from the interactions, as recurring interaction motifs can characterize specific cellular behaviors. The control unit (130) may be further configured to classify the interaction as following a particular interaction pattern, enabling identification and quantification of distinct interaction types. The classification may be performed by machine learning, which excels at finding patterns in complex interaction data. The classification may comprise one or more decision-making method selected from statistical classification techniques, decision trees, rule-based systems, Bayesian methods, ensemble methods, support vector machines, nearest neighbor, clustering techniques, dimensionality reduction techniques, amongst others.

In embodiments, the control unit (130) may be further configured to detect changes in the biological cell (2) features, which is key to assessing cell responses. The control unit (130) may be further configured to quantify the changes in the biological cell (2) features, allowing precise comparisons between conditions.

In embodiments, at least one image every 30 seconds, for example at least one image every 20 seconds may be captured during the sequence to enable detecting rapid cellular events. For instance, at least one image every 10 seconds or at least one image every 5 seconds, 1 second, or even 1 millisecond may be captured.

In embodiments, the plurality of discrete confinement locations (110) may be an array of microcavities (210) or wherein the containment unit comprises a microfluidic unit (200) configured for forming an array of droplets (220), the microcavities (210) or droplets (220) being for biological cell (2) holding. The droplets (220) may for instance be aqueous droplets either in air or in oil.

In embodiments, the containment unit (100) (e.g., the microfluidic unit (200)) may be adapted for enabling the establishment of different cell-stimuli conditions, allowing testing of cells under various stimuli in parallel. The different cell-stimuli conditions may be different biological cell environments, e.g., different stimuli or different stimuli concentration, in different confinement locations (110) amongst the plurality of the containment unit (100). The different cell-stimuli conditions may be different types and/or different ratio of biological cells (2) in different confinement locations (110) amongst the plurality of the containment unit (100).

In embodiments, the system (1) may further comprise a fluidic control system for introducing or removing fluids from the containment unit (100), enabling dynamic stimulation of the cells (2).

In embodiments, the plurality of discrete confinement locations (110) may be at least 20, for example at least 200, for example at least 2000, for example at least 20000, for example at least 200000 locations to increase the throughput and statistical power.

In embodiments, the lens-free imaging module (120) may be configured to perform holographic imaging to provide 3D information about the cells (2).

In embodiments, the system (1) may further comprise at least one biological cell (2) in one of the confinement locations (110) to enable the analysis.

In the second aspect, the present disclosure relates to a method for monitoring biological cells (2), the method comprising:
a. Arranging biological cells (2) within a containment unit (100), the containment unit (100) being structured to maintain the biological cells (2) in a plurality of discrete confinement locations (110);
b. Employing a lens-free imaging module (120) to capture a plurality of images, wherein each image simultaneously depicts all biological cells (2) positioned within the containment unit (100);
c. Operating the lens-free imaging module (120) to capture a sequence of the images over a period of time; and
d. Extract from the images biological cell (2) features.

FIG. 2 presents a flowchart illustrating the method for monitoring biological cells (2) using the system (1) according to embodiments of the present disclosure. The flowchart begins with the step of arranging cells (2) within discrete locations (110) of a containment unit (100). Following this, the lens-free imaging module (120) is employed to capture images of the cells (2). The next step involves operating the imaging module (120) to capture a sequence of images over time, allowing for continuous monitoring of the cells (2). The final step in the flowchart is the extraction of cell (2) features from these images, which is beneficial for analyzing and characterizing the biological cells (2) based on the captured date, e.g., time-lapse data.

In embodiments, step a may include arranging biological cells (2) within the containment unit (100) so that at least two biological cells (2) are in two different discrete confinement locations (110) differing in their cell-stimuli conditions, enabling comparison of cell responses to different stimuli. The different cell-stimuli conditions may be different biological cell environments, e.g., different stimuli or different stimuli concentration. The different cell-stimuli conditions may be different types and/or different ratio of biological cells (2).

In the third aspect, the present disclosure relates to a computer program comprising instructions to cause the system (1) of the first aspect to execute the steps of the method of the second aspect.

In the fourth aspect, the present disclosure relates to a computer-readable medium having stored thereon the computer program of the third aspect.

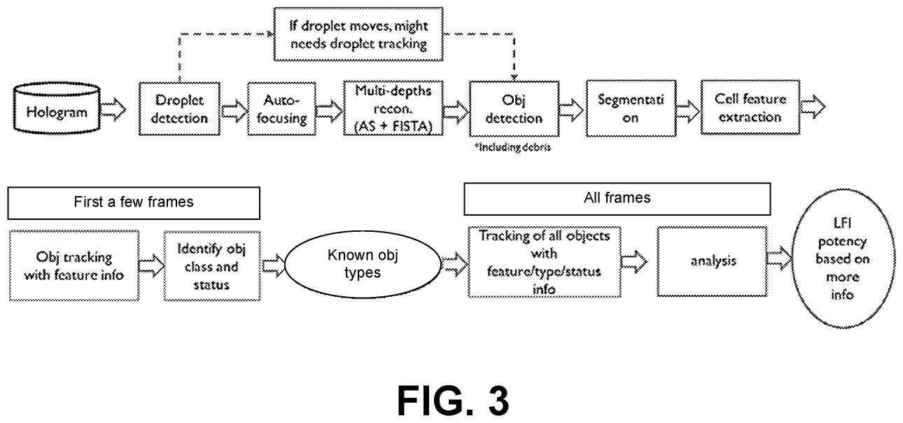

FIG. 3 illustrates an example of algorithm pipeline that can be used in the system (1) for biological cell (2) monitoring. The pipeline begins with the generation of a hologram. Following this, droplet (220) detection is performed to identify and track the position of each droplet (220), which can provide additional benefits if the droplet (220) moves, implicating droplet (220) tracking. Auto-focusing is integrated to ensure clarity and precision in the imaging process, followed by multi-depths reconstruction (using algorithms such as AS+FISTA) to accurately reconstruct the image from the hologram.

The next phase involves object detection, which includes identifying debris along with the biological cells (2). This leads to the segmentation process, which can be beneficial for distinguishing individual cells (2) or objects within the droplet (220). Cell (2) feature extraction follows, where specific features of the cells (2) are identified and recorded.

The initial few frames focus on object tracking with feature information, identifying object class and status, and recognizing object types. As the process continues through all frames, tracking of all objects with their respective feature/type/status information is maintained.

Finally, the analysis phase uses the accumulated data to assess the potency based on more comprehensive information gathered throughout the imaging and tracking process. This detailed analysis allows for a thorough understanding of cell (2) behavior and interactions over time.

### Example 1: Massive Cell Characterization by Time-Lapse Cell-Cell Interaction Monitoring

An experiment is conducted to characterize biological cells by monitoring their interactions with stimuli over time using a lens-free imaging system. The aim of the experiment is to accurately assess cell response and activity by capturing comprehensive time-lapse data, overcoming the limitations of snapshot imaging approaches.

The experimental setup includes a lens-free imaging system with a light source and a plurality of imagers, and a fluidic device with transparent compartments for confining cells. Although a plurality of imagers is used here, a single imager can be used as well. Biological cells and stimuli are distributed in the compartments, either deterministically or randomly. The lens-free imaging allows for massively parallel monitoring of cell-cell interaction events across a large field of view with high time resolution, capturing fast cellular events such as cell response to stimuli. The segmentation of cells in isolated compartments enables heterogeneous assays and prevents cell aggregate formation, facilitating the identification and tracking of individual cells.

The time-lapse method involves identifying and tracking cells per time frame, assigning each cell a unique identifier. Cell morphology features, including static features like cell size, circularity, nucleus/cell size ratio, and optical intensity contrast between different parts of cells, as well as dynamic features like cell motility, are extracted. Cell-cell interaction patterns, such as the frequency and extent of cell conjugation, are also analyzed and assigned event IDs. The cell features and interaction patterns are compared across multiple time frames for each cell and event, allowing for comprehensive characterization of cells and their activities.

The experiment demonstrated the effectiveness of using dynamic changes in cell features over time and cell-cell interaction patterns to characterize cells. In a typical embodiment of the cell response assay, biological cells are encapsulated with stimulus cells in droplets or micro wells at controlled or stochastic ratios. The interactions between biological and stimulus cells are monitored by lens-free imaging, measuring cell feature changes to derive the cell response efficacy. This process is repeated for positive and negative control samples, using standard responsive and non-responsive cells, respectively, with the same stimulus. The cell response efficacy derived from all three samples is compared to determine the cell response potency of the biological cell product.

The results shows that the time-lapse lens-free imaging approach provided beneficial time-domain information for extracting cell-cell interaction patterns and characterizing cells, such as their response potency. This label-free method proves to be valuable for cell characterizations in applications like cell therapy and drug development, offering improvements over fluorescence cell imaging, flow cell cytometry, and ELISA assays in terms of convenience, real-time monitoring, and the ability to capture comprehensive cell activity data.

This example demonstrates the effectiveness of using time-lapse cell-cell interaction monitoring with lens-free imaging for massive cell characterization. The approach overcomes the limitations of snapshot imaging and provides a powerful tool for accurately assessing cell response and activity in various applications.

### Example 2: Monitoring Cell-Cell Interactions

The system of example 1 is used to monitor the interaction between two types of cell. The containment unit comprises an array of 10,000 microwells, each holding both types of cell. In some embodiments, the microfluidic device has an array of at least 10,000 microwells, or even at least 100,000 microwells. The lens-free imaging module captures images of the entire array every 10 seconds for a total duration of 2 hours. The control unit extracts cell motility and cell-cell contact duration features from the image sequence. It classifies the cell response as strong, moderate, or weak based on the extracted features using a decision tree algorithm.

### Example 3: Stem Cell Differentiation Assay with Varying Factor Concentrations

The system is configured with a microfluidic device that generates 500,000 aqueous droplets in oil. Each droplet contains a single stem cell and a differentiation factor at varying concentrations. The lens-free imaging module captures images of the droplets every 15 seconds for 12 hours. The control unit measures changes in cell morphology features like cell size and circularity over time. It quantifies the stem cell differentiation efficiency under different factor concentrations.

### Example 4: Analyzing Neuron-Oligodendrocyte Interactions During Myelination

The containment unit is a microfluidic chip with 200 chambers. Each chamber is loaded with neurons and oligodendrocyte precursor cells at different ratios. The lens-free imaging module acquires images every 20 seconds for 24 hours. The control unit tracks individual cells and extracts cell migration trajectories. It detects myelin sheath formation by analyzing the interaction patterns between neurons and oligodendrocytes using a convolutional neural network.

### Example 5: Bacteria Cell Characterizations and Antibiotics Susceptibility Testing

The system monitors the effect of different antibiotics on bacterial cells. The containment unit has 500 to 50,000 microwells, each containing a single or multiple bacterial cell. Typically the number of microwells is determined by the assay. For example, in antimicrobial susceptibility testing (AST), the number of micro wells are determined by the number of antibiotics multiplied by the number of concentrations (or titrations) of each antibiotics. The typical number is several hundred but can also be thousands. Here, different antibiotics at various concentrations are introduced into the wells using a fluidic control system. The lens-free imaging module takes images every 5 seconds for 1 hour. The control unit measures changes in bacterial cell morphology, the bacteria motility patterns, and the proliferation patterns such as the colony formation. Then the control unit identifies the bacteria type based on these feature and feature changes. Likewise the control unit also determines the most effective antibiotics or the antibiotics spectrum. These can be done by employing image pattern recognition techniques, for example, if necessary machine learning techniques.

### Example 6: Studying Macrophage Interactions With Surface-Modified Nanoparticles

The system is applied to study the interaction between macrophages and nanoparticles. The containment unit is a microfluidic device that creates an array of 100,000 droplets, each encapsulating a single macrophage and nanoparticles with different surface modifications. The lens-free imaging module captures images every 30 seconds for 6 hours. The control unit analyzes the nanoparticle uptake by macrophages over time and quantifies the effect of surface modifications on the phagocytosis process.

It is to be understood that although example embodiments, specific constructions and configurations, as well as materials, have been discussed herein for devices according to the present disclosure, various changes or modifications in form and detail may be made without departing from the scope of subject matter of the present disclosure. For example, any formulas given above are merely representative of procedures that may be used. Functionality may be added or deleted from the block diagrams and operations may be interchanged among functional blocks. Steps may be added or deleted to methods described within the scope of the present disclosure.

### Example 7: Cell Marker Characterizations

The system monitors the interactions between cells and micro or sub-micrometer features. The features can be micro or sub-micrometer structures or particles. The structures can be a pre-defined region, a pillars, holes in a micro cavity. The structures or particles are coated with molecules targeting specific cell markers on the cell membrane. The cell-structure or cell-particle interactions are thus mediated by the affinity-based molecule recognitions. The interaction frequency and duration indicate the affinity and quantity of the cell markers.

In this case there is at least one cell and one or more structure or particle in a micro confinement. There can be multiple types of cells per assay. Different cell types can be loaded in separate confinements for independent cell marker characterizations, or they can be co-loaded in a same confinement for cell marker investigations before/during/after the cell-cell interactions. There can be multiple types of structures or particles. This is particularly interesting for multiplexed marker characterizations. Each structure or particle type is coated by a unique type of molecule or molecule mixture. The structure or particle types can differ in size, shape, density or the optical refractive index. All these can be distinguished by LFI. The structure can also differ by location.

Depending on the precision, speed and multiplexing requirements of the cell marker characterizations, the assay may require 100 to 100,000 micro confinements.

In this application, the LFI monitoring is typically very frequent, such as 10 images per seconds, but can also set to 1,000 images per seconds if needed.

1. A system for biological cell monitoring, comprising:
a containment unit comprising a plurality of discrete confinement locations, the containment unit being adapted to hold at least one biological cell per confinement location;
a lens-free imaging module configured to capture holographic images, each holographic image depicting all biological cells within the containment unit , wherein the lens-free imaging module comprises a light source and an imager configured to capture the holographic images using light from the light source, and wherein the light source and imager are disposed on opposite sides of the containment unit; and
a control unit configured to:
operate the lens-free imaging module to capture a sequence of holographic images over a period of time; and
extract, from the holographic images, biological cell features of the biological cells held by the containment unit.

2. The system of claim 1, wherein the control unit is further configured to:
based on an initial subset of the holographic images, tracking a set of objects in the initial subset of the holographic images using the extracted biological cell features and identifying for each object of the set of objects a respective object class, status, and object type; and
based on a subsequent subset of the holographic images, tracking the set of objects in the subsequent subset of the holographic images using the identified object classes, statuses, and object types.

3. The system of claim 1, wherein the control unit is further configured to detect and classify the individual biological cell from the biological cell features.

4. The system of claim 1, wherein the control unit is further configured to detect an interaction of the biological cell with a stimulus based on an evolution of the biological cell features in the sequence.

5. The system of claim 1, wherein the containment unit comprises a microfluidic unit configured to form an array of droplets that hold the biological cells.

6. The system of claim 4, wherein the control unit is further configured to determine an interaction pattern from the interaction.

7. The system of claim 1, wherein the control unit is further configured to detect changes in the biological cell features.

8. The system of claim 1, wherein the containment unit comprises a two-dimensional array of at least 10,000 confinement locations.

9. The system of claim 1, wherein the plurality of discrete confinement locations are an array of microcavities configured to hold biological cells.

10. The system of claim 1, wherein the control unit is further configured to:
from each holographic image, reconstruct two or more images at respective two or more different depths.

11. The system of claim 10, wherein reconstructing two or more images at respective two or more different depths comprises applying the architecture-searched fast iterative shrinkage thresholding algorithm to at least one of the holographic images.

12. A method for monitoring biological cells, the method comprising:
arranging biological cells within a containment unit, the containment unit being structured to maintain the biological cells in a plurality of discrete confinement locations;
using a lens-free imaging module that is configured to capture holographic images to capture a plurality of holographic images, wherein each holographic image simultaneously depicts all biological cells positioned within the containment unit , wherein the lens-free imaging module comprises a light source and an imager configured to capture the holographic images using light from the light source, and wherein the light source and imager are disposed on opposite sides of the containment unit;
operating the lens-free imaging module to capture a sequence of the holographic images over a period of time; and
extracting from the holographic images biological cell features of the biological cells held by the containment unit.

13. The method of claim 12, wherein arranging the biological cells within the containment unit comprises arranging biological cells within the containment unit so that at least two biological cells are in two different discrete confinement locations differing in their cell-stimuli conditions.

14. (canceled)

15. A computer-readable medium having stored thereon program instructions that, when executed by a controller, cause to controller to perform controller operations comprising:
using a lens-free imaging module that is configured to capture holographic images to capture a plurality of holographic images, wherein each holographic image simultaneously depicts all biological cells positioned within a containment unit, wherein the lens-free imaging module comprises a light source and an imager configured to capture the holographic images using light from the light source, wherein the light source and imager are disposed on opposite sides of the containment unit, and wherein the containment unit is structured to maintain the biological cells in a plurality of discrete confinement locations;
operating the lens-free imaging module to capture a sequence of the holographic images over a period of time; and
extracting from the holographic images biological cell features of the biological cells held by the containment unit.

16. The method of claim 12, further comprising:
based on an initial subset of the holographic images, tracking a set of objects in the initial subset of the holographic images using the extracted biological cell features and identifying for each object of the set of objects a respective object class, status, and object type; and
based on a subsequent subset of the holographic images, tracking the set of objects in the subsequent subset of the holographic images using the identified object classes, statuses, and object types.

17. The method of claim 12, wherein the containment unit comprises a microfluidic unit configured to form an array of droplets that hold the biological cells.

18. The method of claim 12, wherein the containment unit comprises a two-dimensional array of at least 10,000 confinement locations.

19. The method of claim 12, wherein the plurality of discrete confinement locations are an array of microcavities configured to hold biological cells.

20. The method of claim 12, further comprising:
from each holographic image, reconstruct two or more images at respective two or more different depths.

21. The system of claim 4, wherein the stimulus comprises at least one of a bead coated with biological molecules or a microstructure coated with biological molecules.
